(19) United States
(12) Patent Application Publication
(10) Pub. No.: US 2024/0122906 A1
(43) Pub. Date: Apr. 18, 2024

(54) METHODS OF TREATING MELANOMA WITH RIPRETINIB

(71) Applicant: Deciphera Pharmaceuticals, LLC, Waltham, MA (US)

(72) Inventors: Ying Su, Waltham, MA (US); Rodrigo Ruiz Soto, Waltham, MA (US)

(21) Appl. No.: 18/505,396

(22) Filed: Nov. 9, 2023

Related U.S. Application Data
(63) Continuation of application No. PCT/US2022/028456, filed on May 10, 2022.
(60) Provisional application No. 63/313,570, filed on Feb. 24, 2022.
(60) Provisional application No. 63/231,384, filed on Aug. 10, 2021.
(60) Provisional application No. 63/187,903, filed on May 12, 2021.

(51) Int. Cl. A61K 31/4375; A61P 35/00

(52) U.S. Cl. CPC A61K 31/4375; A61P 35/00

(57) ABSTRACT

The present disclosure relates to methods of treating melanoma in a patient in need thereof, comprising administering to the subject a therapeutically effective amount of ripretinib or a pharmaceutically acceptable salt thereof.

### CROSS-REFERENCE

This application is a continuation of International Application Serial No. PCT/US2022/028456 filed May 10, 2022, which claims priority to U.S. Provisional Application No. 63/187,903 filed May 12, 2021, U.S. Provisional Application No. 63/231,384 filed Aug. 10, 2021, and U.S. Provisional Application No. 63/313,570 filed Feb. 24, 2022, the contents of each of which are incorporated herein by reference in their entireties.

### BACKGROUND

c-KIT (also known as KIT, CD117, and stem cell factor receptor) is a 145 kDa transmembrane tyrosine kinase protein that acts as a type-III receptor. The c-KIT proto-oncogene, located on chromosome 4q11-21, encodes the c-KIT receptor, whose ligand is the stem cell factor (SCF), steel factor, kit ligand, and mast cell growth factor. The receptor has tyrosine-protein kinase activity and binding of the ligand SCF leads to the autophosphorylation of c-KIT and its association with substrates such as phosphatidylinositol 3-kinase (PI3K) activating the PI3K/AKT signaling pathway and also activating the RAS/MAPK signaling pathway through RAF, MEK, and ERK kinases. Oncogenic mutations in cKIT or overexpression of wildtype KIT leads to dysregulation of KIT signaling, enabling uncontrolled activation of cKIT independent of control by activating ligands such as SCF. Tyrosine phosphorylation by protein tyrosine kinases is of particular importance in cellular signaling and can mediate signals for major cellular processes, such as proliferation, survival, differentiation, apoptosis, attachment, invasiveness and migration. Defects in c-KIT are a cause of piebaldism, an autosomal dominant genetic developmental abnormality of pigmentation characterized by congenital patches of white skin and hair that lack melanocytes. Activating mutations in the receptor tyrosine kinase KIT have been identified in multiple cancer types such as melanoma. In addition, aberrant wild-type KIT overexpression is found in melanoma.

A need exists for therapeutics that can broadly inhibit clinically relevant KIT mutations for the treatment of melanoma.

### SUMMARY

Described herein, in part, are methods of treating melanoma in a patient in need thereof comprising administering to the patient a therapeutically effective amount of ripretinib or a pharmaceutically acceptable salt thereof.

In one embodiment, described herein is a method of treating a KIT driven melanoma in a patient in need thereof, comprising orally administering to the patient 100 mg to 600 mg of ripretinib daily.

In another embodiment, described herein is a method of treating a KIT driven melanoma in a patient in need thereof, comprising orally administering to the patient one or more tablets comprising 100 mg to 600 mg ripretinib daily.

In another embodiment, described herein is a method of treating a KIT driven melanoma in a patient in need thereof, comprising orally administering to the patient 100 mg to 600 mg of ripretinib daily, wherein the patient has not been previously administered one or more tyrosine kinase inhibitors before administration of the ripretinib.

In another embodiment, described herein is a method of treating KIT driven melanoma in a patient in need thereof, comprising orally administering to the patient 100 mg to 600 mg of ripretinib daily, wherein the patient was previously administered at least one tyrosine kinase inhibitor before administration of the ripretinib.

In one embodiment, described herein is a method of treating KIT driven melanoma in a patient in need thereof, comprising orally administering to the patient, on a daily basis, one or more tablets each comprising ripretinib, e.g., tablets each comprising 50 mg to 100 mg of ripretinib, wherein the patient was previously administered at least one tyrosine kinase inhibitor before administration of the ripretinib.

In one embodiment, described herein is a method of treating melanoma in a patient in need thereof, comprising orally administering to the patient 100 mg to 600 mg of ripretinib daily, and one or more additional therapeutic agents.

### BRIEF DESCRIPTION OF THE DRAWINGS

FIG. 1 depicts an exemplary plot of survival probability with respect to progression-free survival (PFS) for KIT driven melanoma patients on ripretinib.

FIG. 2 depicts exemplary plots illustrating progression-free survival (PFS) among KIT driven melanoma patients receiving ripretinib who did not receive prior TKI treatment (“Non-TKI”) and who did receive prior TKI treatment (“TKI”).

FIG. 3 shows duration of treatment with ripretinib in melanoma patients in the study of Example 1.

FIG. 4 depicts best overall response in individual patients and their respective percentage changes of target lesion from baseline after ripretinib treatment from the study of Example 1. Abbreviations: PD: progressive disease; SD: stable disease; PR: partial response; CR: complete response.

FIG. 5 depicts a spider plot of changes in target lesion in individual patients with respect to treatment duration from the study of Example 1.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

As used herein, “ripretinib” is a compound represented by the following structure:

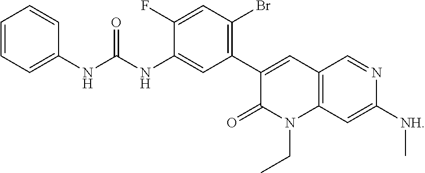 [SMILES: CCn1c(=O)c(-c2cc(NC(=O)Nc3ccccc3)c(F)cc2Br)cc2cnc(NC)cc21]

As used herein, “Compound A” is a compound represented by the following structure:

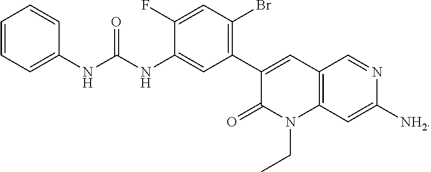 [SMILES: CCn1c(=O)c(-c2cc(NC(=O)Nc3ccccc3)c(F)cc2Br)cc2cnc(N)cc21]

“Individual,” “patient,” or “subject” are used interchangeably herein and include any animal, including mammals, including mice, rats, other rodents, rabbits, dogs, cats, swine, cattle, sheep, horses, or primates, and humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods described herein is desirably a mammal in which treatment of a disorder described herein is desired, such as a human.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

As used herein, “treating” includes any effect, e.g., lessening, reducing, modulating, or eliminating, that results in the improvement of the condition, disease, disorder and the like.

“Therapeutically effective amount” includes the amount of the subject compound that will elicit the biological or medical response of a tissue, system, animal or human that is being sought by the researcher, veterinarian, medical doctor or other clinician. A compound described herein, e.g., ripretinib is administered in therapeutically effective amounts to treat a condition described herein, e.g., melanoma. Alternatively, a therapeutically effective amount of a compound is the quantity required to achieve a desired therapeutic and/or prophylactic effect, such as an amount which results in the prevention of or a decrease in the symptoms associated with the condition.

As used herein, “AUC₀₋₂₄ₕ” refers to the area under the plasma concentration-time curve from time zero to 24 hours for a compound described herein. As used herein, “AUC_{0-inf}” refers to the area under the plasma concentration-time curve from time zero to infinite time for a compound described herein. As used herein, “Cₘₐₓ” refers to the maximum plasma concentration of a compound described herein.

A compound described herein, e.g., ripretinib, can be formulated as a pharmaceutical composition using a pharmaceutically acceptable carrier and administered by a variety of routes. In some embodiments, such compositions are for oral administration. In some embodiments, compositions formulated for oral administration are provided as tablets. In some embodiments, such compositions are for parenteral (by injection) administration (e.g., a composition formulated for local injection at the site of a tumor, e.g., a diffuse-type giant cell tumor). In some embodiments, such compositions are for transdermal administration. In some embodiments, such compositions are for topical administration. In some embodiments, such compositions are for intravenous (IV) administration. In some embodiments, such compositions are for intramuscular (IM) administration. Such pharmaceutical compositions and processes for preparing them are well known in the art. See, e.g., REMINGTON: THE SCIENCE AND PRACTICE OF PHARMACY (A. Gennaro, et al., eds., 19^{th} ed., Mack Publishing Co., 1995).

A “combination therapy” is a treatment that includes the administration of two or more therapeutic agents, to a patient. The two or more therapeutic agents may be delivered at the same time, e.g., in separate pharmaceutical compositions or in the same pharmaceutical composition, or they may be delivered at different times. For example, they may be delivered concurrently or during overlapping time periods, and/or one therapeutic agent may be delivered before or after the other therapeutic agent(s). Treatment with a combination therapy optionally includes treatment with either single agent, preceded or followed by a period of concurrent treatment with both agents. However, it is contemplated that during some time period, effective amounts of the two or more therapeutic agents are present within the patient.

### Methods of Treatment

Described herein are methods of treating KIT driven melanoma in a patient in need thereof. In an embodiment, provided herein is a method of treating a patient suffering from melanoma, comprising administering to the patient a therapeutically effective amount of ripretinib or a pharmaceutically acceptable salt thereof.

A melanoma described herein may be a cutaneous melanoma or noncutaneous melanoma. In some embodiments, a melanoma described herein is a cutaneous melanoma. In some embodiments, the cutaneous melanoma is selected from the group consisting of superficial spreading melanoma, nodular melanoma, acral-lentiginous melanoma, and amelanotic and desmoplastic melanoma. In some embodiments, a melanoma described herein is a noncutaneous (non-skin) melanoma. In some embodiments, the noncutaneous melanoma is selected from ocular melanoma and mucosal melanoma. In some embodiments, the noncutaneous melanoma is ocular melanoma. In embodiments, the noncutaneous melanoma is mucosal melanoma. In some embodiments, a melanoma described herein is acral-lentiginous melanoma. In some embodiments, a melanoma described herein is related to chronic sun damage on the patient's skin.

In one embodiment, the present disclosure provides a method of treating melanoma in a patient in need thereof, comprising administering, e.g., orally administering, to the patient 100 mg or more of ripretinib daily, e.g., 100 mg to 5000 mg, e.g., 100 mg to 600 mg, e.g., 100 mg to 500 mg, e.g., 100 mg to 300 mg, e.g., 100 mg to 250 mg, e.g., 150 mg. In some embodiments, the melanoma is a KIT activated melanoma. In some embodiments, the KIT activated melanoma has a baseline mutation selected from the group consisting of a KIT exon 9 mutation, a KIT exon 11 mutation, a KIT exon 13 mutation, a KIT exon 17 mutation, and a KIT exon 18 mutation. In some embodiments, after at least one 42-28-day cycle, the patient has a progression-free survival as measured using RECIST 1.1. In some embodiments, the method comprises administering to the patient 110 mg of ripretinib daily. In some embodiments, the method comprises administering to the patient 120 mg of ripretinib daily. In some embodiments, the method comprises administering to the patient 130 mg of ripretinib daily. In some embodiments, the method comprises administering to the patient 140 mg of ripretinib daily. In some embodiments, the method comprises administering to the patient 150 mg of ripretinib daily. In some embodiments, the method comprises administering to the patient 200 mg of ripretinib daily. In some embodiments, the method comprises administering to the patient 250 mg of ripretinib daily. In some embodiments, the method comprises administering to the patient 300 mg of ripretinib daily. In some embodiments, the method comprises administering to the patient 350 mg of ripretinib daily. In some embodiments, the method comprises administering to the patient 400 mg of ripretinib daily. In some embodiments, the method comprises administering to the patient 450 mg of ripretinib daily. In some embodiments, the method comprises administering to the patient 500 mg of ripretinib daily. In some embodiments, the method comprises administering to the patient 550 mg of ripretinib daily. In some embodiments, the method comprises administering to the patient 600 mg of ripretinib daily. In some embodiments, the method comprises administering to the patient 650 mg of ripretinib daily. In some embodiments, the method comprises administering to the patient 700 mg of ripretinib daily. In some embodiments, the method comprises administering to the patient 750 mg of ripretinib daily. In some embodiments, the method comprises administering to the patient 800 mg of ripretinib daily. In some embodiments, the method comprises administering to the patient 850 mg of ripretinib daily. In some embodiments, the method comprises administering to the patient 900 mg of ripretinib daily. In some embodiments, the method comprises administering to the patient 950 mg of ripretinib daily. In some embodiments, the method comprises administering to the patient 1000 mg of ripretinib daily. In some embodiments, the method comprises administering to the patient 150 mg of ripretinib once daily. In some embodiments, the method comprises administering to the patient 200 mg of ripretinib once daily. In some embodiments, the method comprises administering to the patient 250 mg of ripretinib once daily. In some embodiments, the method comprises administering to the patient 300 mg of ripretinib once daily. In some embodiments, the method comprises administering to the patient 350 mg of ripretinib once daily. In some embodiments, the method comprises administering to the patient 400 mg of ripretinib once daily. In some embodiments, the method comprises administering to the patient 450 mg of ripretinib daily. In some embodiments, the method comprises administering to the patient 500 mg of ripretinib once daily. In some embodiments, the method comprises administering to the patient 550 mg of ripretinib once daily. In some embodiments, the method comprises administering to the patient 600 mg of ripretinib once daily. In some embodiments, the method comprises administering to the patient 650 mg of ripretinib once daily. In some embodiments, the method comprises administering to the patient 700 mg of ripretinib once daily. In some embodiments, the method comprises administering to the patient 750 mg of ripretinib once daily. In some embodiments, the method comprises administering to the patient 800 mg of ripretinib once daily. In some embodiments, the method comprises administering to the patient 850 mg of ripretinib once daily. In some embodiments, the method comprises administering to the patient 900 mg of ripretinib once daily. In some embodiments, the method comprises administering to the patient 950 mg of ripretinib once daily. In some embodiments, the method comprises administering to the patient 1000 mg of ripretinib once daily. In some embodiments, the method comprises administering to the patient 150 mg of ripretinib twice daily. In some embodiments, the method comprises administering to the patient 200 mg of ripretinib twice daily. In some embodiments, the method comprises administering to the patient 250 mg of ripretinib twice daily. In some embodiments, the method comprises administering to the patient 300 mg of ripretinib twice daily. In some embodiments, the method comprises administering to the patient 350 mg of ripretinib twice daily. In some embodiments, the method comprises administering to the patient 400 mg of ripretinib twice daily. In some embodiments, the method comprises administering to the patient 450 mg of ripretinib twice daily. In some embodiments, the method comprises administering to the patient 500 mg of ripretinib twice daily.

In one embodiment, the present disclosure provides a method of treating melanoma in a patient in need thereof, comprising administering, e.g., orally administering, to the patient 100 mg or more of ripretinib daily, e.g., 100 mg to 5000 mg, e.g., 100 mg to 600 mg, e.g., 100 mg to 500 mg, e.g., 100 mg to 300 mg, e.g., 100 mg to 250 mg, e.g., 150 mg. In some embodiments, the melanoma is a KIT driven melanoma. In some embodiments, the KIT driven melanoma has a baseline mutation selected from the group consisting of a KIT exon 9 mutation, a KIT exon 11 mutation, a KIT exon 13 mutation, a KIT exon 17 mutation, and a KIT exon 18 mutation. In some embodiments, the KIT driven melanoma is caused by overexpression of wildtype KIT. In some embodiments, after at least one 28-day cycle, the patient has a progression-free survival as measured using RECIST 1.1. In some embodiments, the method comprises administering to the patient 110 mg of ripretinib daily. In some embodiments, the method comprises administering to the patient 120 mg of ripretinib daily. In some embodiments, the method comprises administering to the patient 130 mg of ripretinib daily. In some embodiments, the method comprises administering to the patient 140 mg of ripretinib daily. In some embodiments, the method comprises administering to the patient 150 mg of ripretinib daily. In some embodiments, the method comprises administering to the patient 200 mg of ripretinib daily. In some embodiments, the method comprises administering to the patient 250 mg of ripretinib daily. In some embodiments, the method comprises administering to the patient 300 mg of ripretinib daily. In some embodiments, the method comprises administering to the patient 350 mg of ripretinib daily. In some embodiments, the method comprises administering to the patient 400 mg of ripretinib daily. In some embodiments, the method comprises administering to the patient 450 mg of ripretinib daily. In some embodiments, the method comprises administering to the patient 500 mg of ripretinib daily. In some embodiments, the method comprises administering to the patient 550 mg of ripretinib daily. In some embodiments, the method comprises administering to the patient 600 mg of ripretinib daily. In some embodiments, the method comprises administering to the patient 650 mg of ripretinib daily. In some embodiments, the method comprises administering to the patient 700 mg of ripretinib daily. In some embodiments, the method comprises administering to the patient 750 mg of ripretinib daily. In some embodiments, the method comprises administering to the patient 800 mg of ripretinib daily. In some embodiments, the method comprises administering to the patient 850 mg of ripretinib daily. In some embodiments, the method comprises administering to the patient 900 mg of ripretinib daily. In some embodiments, the method comprises administering to the patient 950 mg of ripretinib daily. In some embodiments, the method comprises administering to the patient 1000 mg of ripretinib daily. In some embodiments, the method comprises administering to the patient 150 mg of ripretinib once daily. In some embodiments, the method comprises administering to the patient 200 mg of ripretinib once daily. In some embodiments, the method comprises administering to the patient 250 mg of ripretinib once daily. In some embodiments, the method comprises administering to the patient 300 mg of ripretinib once daily. In some embodiments, the method comprises administering to the patient 350 mg of ripretinib once daily. In some embodiments, the method comprises administering to the patient 400 mg of ripretinib once daily. In some embodiments, the method comprises administering to the patient 450 mg of ripretinib daily. In some embodiments, the method comprises administering to the patient 500 mg of ripretinib once daily. In some embodiments, the method comprises administering to the patient 550 mg of ripretinib once daily. In some embodiments, the method comprises administering to the patient 600 mg of ripretinib once daily. In some embodiments, the method comprises administering to the patient 650 mg of ripretinib once daily. In some embodiments, the method comprises administering to the patient 700 mg of ripretinib once daily. In some embodiments, the method comprises administering to the patient 750 mg of ripretinib once daily. In some embodiments, the method comprises administering to the patient 800 mg of ripretinib once daily. In some embodiments, the method comprises administering to the patient 850 mg of ripretinib once daily. In some embodiments, the method comprises administering to the patient 900 mg of ripretinib once daily. In some embodiments, the method comprises administering to the patient 950 mg of ripretinib once daily. In some embodiments, the method comprises administering to the patient 1000 mg of ripretinib once daily. In some embodiments, the method comprises administering to the patient 150 mg of ripretinib twice daily. In some embodiments, the method comprises administering to the patient 200 mg of ripretinib twice daily. In some embodiments, the method comprises administering to the patient 250 mg of ripretinib twice daily. In some embodiments, the method comprises administering to the patient 300 mg of ripretinib twice daily. In some embodiments, the method comprises administering to the patient 350 mg of ripretinib twice daily. In some embodiments, the method comprises administering to the patient 400 mg of ripretinib twice daily. In some embodiments, the method comprises administering to the patient 450 mg of ripretinib twice daily. In some embodiments, the method comprises administering to the patient 500 mg of ripretinib twice daily.

In an embodiment, a method of treating a patient suffering from melanoma, comprising administering, e.g., orally administering, to the patient 100 mg or more of ripretinib daily, e.g., up to about 600 mg, e.g., 100 mg to 250 mg, e.g., 100 mg to 500 mg, e.g., 100 mg to 300 mg, e.g., 100 mg to 250 mg, e.g., 150 mg, wherein the patient was previously administered at least one tyrosine kinase inhibitor, is contemplated. In some embodiments, the melanoma is a KIT driven melanoma. In some embodiments, the KIT activated melanoma has a baseline mutation selected from the group consisting of a KIT exon 9 mutation, a KIT exon 11 mutation, a KIT exon 13 mutation, a KIT exon 17 mutation, and a KIT exon 18 mutation. In some embodiments, the KIT driven melanoma is caused by overexpression of wildtype KIT. In some embodiments, the at least one previously administered tyrosine kinase inhibitor is selected from the group consisting of imatinib, sunitinib, regorafenib, nilotinib, avapritinib, and AZD3229, and pharmaceutically acceptable salts thereof. In some embodiments, the patient has previously been administered two, three, four, or five separate tyrosine kinase inhibitors. In some embodiments, after at least one 28-day cycle, the patient has a progression-free survival as measured using RECIST 1.1. In some embodiments, the method comprises administering to the patient 110 mg of ripretinib daily. In some embodiments, the method comprises administering to the patient 120 mg of ripretinib daily. In some embodiments, the method comprises administering to the patient 130 mg of ripretinib daily. In some embodiments, the method comprises administering to the patient 140 mg of ripretinib daily. In some embodiments, the method comprises administering to the patient 150 mg of ripretinib daily. In some embodiments, the method comprises administering to the patient 200 mg of ripretinib daily. In some embodiments, the method comprises administering to the patient 250 mg of ripretinib daily. In some embodiments, the method comprises administering to the patient 300 mg of ripretinib daily. In some embodiments, the method comprises administering to the patient 350 mg of ripretinib daily. In some embodiments, the method comprises administering to the patient 400 mg of ripretinib daily. In some embodiments, the method comprises administering to the patient 450 mg of ripretinib daily. In some embodiments, the method comprises administering to the patient 500 mg of ripretinib daily. In some embodiments, the method comprises administering to the patient 550 mg of ripretinib daily. In some embodiments, the method comprises administering to the patient 600 mg of ripretinib daily. In some embodiments, the method comprises administering to the patient 650 mg of ripretinib daily. In some embodiments, the method comprises administering to the patient 700 mg of ripretinib daily. In some embodiments, the method comprises administering to the patient 750 mg of ripretinib daily. In some embodiments, the method comprises administering to the patient 800 mg of ripretinib daily. In some embodiments, the method comprises administering to the patient 850 mg of ripretinib daily. In some embodiments, the method comprises administering to the patient 900 mg of ripretinib daily. In some embodiments, the method comprises administering to the patient 950 mg of ripretinib daily. In some embodiments, the method comprises administering to the patient 1000 mg of ripretinib daily. In some embodiments, the method comprises administering to the patient 100 mg of ripretinib once daily. In some embodiments, the method comprises administering to the patient 150 mg of ripretinib once daily. In some embodiments, the method comprises administering to the patient 200 mg of ripretinib once daily. In some embodiments, the method comprises administering to the patient 250 mg of ripretinib once daily. In some embodiments, the method comprises administering to the patient 300 mg of ripretinib once daily. In some embodiments, the method comprises administering to the patient 350 mg of ripretinib once daily. In some embodiments, the method comprises administering to the patient 400 mg of ripretinib once daily. In some embodiments, the method comprises administering to the patient 450 mg of ripretinib daily. In some embodiments, the method comprises administering to the patient 500 mg of ripretinib once daily. In some embodiments, the method comprises administering to the patient 550 mg of ripretinib once daily. In some embodiments, the method comprises administering to the patient 600 mg of ripretinib once daily. In some embodiments, the method comprises administering to the patient 650 mg of ripretinib once daily. In some embodiments, the method comprises administering to the patient 700 mg of ripretinib once daily. In some embodiments, the method comprises administering to the patient 750 mg of ripretinib once daily. In some embodiments, the method comprises administering to the patient 800 mg of ripretinib once daily. In some embodiments, the method comprises administering to the patient 850 mg of ripretinib once daily. In some embodiments, the method comprises administering to the patient 900 mg of ripretinib once daily. In some embodiments, the method comprises administering to the patient 950 mg of ripretinib once daily. In some embodiments, the method comprises administering to the patient 1000 mg of ripretinib once daily. In some embodiments, the method comprises administering to the patient 150 mg of ripretinib twice daily. In some embodiments, the method comprises administering to the patient 200 mg of ripretinib twice daily. In some embodiments, the method comprises administering to the patient 250 mg of ripretinib twice daily. In some embodiments, the method comprises administering to the patient 300 mg of ripretinib twice daily. In some embodiments, the method comprises administering to the patient 350 mg of ripretinib twice daily. In some embodiments, the method comprises administering to the patient 400 mg of ripretinib twice daily. In some embodiments, the method comprises administering to the patient 450 mg of ripretinib twice daily. In some embodiments, the method comprises administering to the patient 500 mg of ripretinib twice daily.

In an embodiment, a method of treating a patient suffering from melanoma, comprising administering, e.g., orally administering, to the patient 100 mg or more of ripretinib daily, e.g., up to about 600 mg, e.g., 100 mg to 250 mg, e.g., 100 mg to 500 mg, e.g., 100 mg to 300 mg, e.g., 100 mg to 250 mg, e.g., 150 mg, wherein the patient has not been previously administered one or more tyrosine kinase inhibitors, is contemplated. In some embodiments, the melanoma is a KIT driven melanoma. In some embodiments, the KIT activated melanoma has a baseline mutation selected from the group consisting of a KIT exon 9 mutation, a KIT exon 11 mutation, a KIT exon 13 mutation, a KIT exon 17 mutation, and a KIT exon 18 mutation. In some embodiments, the KIT driven melanoma is caused by overexpression of wildtype KIT. In some embodiments, the at least one previously administered tyrosine kinase inhibitor is selected from the group consisting of imatinib, sunitinib, regorafenib, nilotinib, avapritinib, and AZD3229, and pharmaceutically acceptable salts thereof. In some embodiments, the patient has previously been administered two, three, four, or five separate tyrosine kinase inhibitors. In some embodiments, after at least one 28-day cycle, the patient has a progression-free survival as measured using RECIST 1.1. In some embodiments, the method comprises administering to the patient 110 mg of ripretinib daily. In some embodiments, the method comprises administering to the patient 120 mg of ripretinib daily. In some embodiments, the method comprises administering to the patient 130 mg of ripretinib daily. In some embodiments, the method comprises administering to the patient 140 mg of ripretinib daily. In some embodiments, the method comprises administering to the patient 150 mg of ripretinib daily. In some embodiments, the method comprises administering to the patient 200 mg of ripretinib daily. In some embodiments, the method comprises administering to the patient 250 mg of ripretinib daily. In some embodiments, the method comprises administering to the patient 300 mg of ripretinib daily. In some embodiments, the method comprises administering to the patient 350 mg of ripretinib daily. In some embodiments, the method comprises administering to the patient 400 mg of ripretinib daily. In some embodiments, the method comprises administering to the patient 450 mg of ripretinib daily. In some embodiments, the method comprises administering to the patient 500 mg of ripretinib daily. In some embodiments, the method comprises administering to the patient 550 mg of ripretinib daily. In some embodiments, the method comprises administering to the patient 600 mg of ripretinib daily. In some embodiments, the method comprises administering to the patient 650 mg of ripretinib daily. In some embodiments, the method comprises administering to the patient 700 mg of ripretinib daily. In some embodiments, the method comprises administering to the patient 750 mg of ripretinib daily. In some embodiments, the method comprises administering to the patient 800 mg of ripretinib daily. In some embodiments, the method comprises administering to the patient 850 mg of ripretinib daily. In some embodiments, the method comprises administering to the patient 900 mg of ripretinib daily. In some embodiments, the method comprises administering to the patient 950 mg of ripretinib daily. In some embodiments, the method comprises administering to the patient 1000 mg of ripretinib daily. In some embodiments, the method comprises administering to the patient 100 mg of ripretinib once daily. In some embodiments, the method comprises administering to the patient 150 mg of ripretinib once daily. In some embodiments, the method comprises administering to the patient 200 mg of ripretinib once daily. In some embodiments, the method comprises administering to the patient 250 mg of ripretinib once daily. In some embodiments, the method comprises administering to the patient 300 mg of ripretinib once daily. In some embodiments, the method comprises administering to the patient 350 mg of ripretinib once daily. In some embodiments, the method comprises administering to the patient 400 mg of ripretinib once daily. In some embodiments, the method comprises administering to the patient 450 mg of ripretinib daily. In some embodiments, the method comprises administering to the patient 500 mg of ripretinib once daily. In some embodiments, the method comprises administering to the patient 550 mg of ripretinib once daily. In some embodiments, the method comprises administering to the patient 600 mg of ripretinib once daily. In some embodiments, the method comprises administering to the patient 650 mg of ripretinib once daily. In some embodiments, the method comprises administering to the patient 700 mg of ripretinib once daily. In some embodiments, the method comprises administering to the patient 750 mg of ripretinib once daily. In some embodiments, the method comprises administering to the patient 800 mg of ripretinib once daily. In some embodiments, the method comprises administering to the patient 850 mg of ripretinib once daily. In some embodiments, the method comprises administering to the patient 900 mg of ripretinib once daily. In some embodiments, the method comprises administering to the patient 950 mg of ripretinib once daily. In some embodiments, the method comprises administering to the patient 1000 mg of ripretinib once daily. In some embodiments, the method comprises administering to the patient 150 mg of ripretinib twice daily. In some embodiments, the method comprises administering to the patient 200 mg of ripretinib twice daily. In some embodiments, the method comprises administering to the patient 250 mg of ripretinib twice daily. In some embodiments, the method comprises administering to the patient 300 mg of ripretinib twice daily. In some embodiments, the method comprises administering to the patient 350 mg of ripretinib twice daily. In some embodiments, the method comprises administering to the patient 400 mg of ripretinib twice daily. In some embodiments, the method comprises administering to the patient 450 mg of ripretinib twice daily. In some embodiments, the method comprises administering to the patient 500 mg of ripretinib twice daily.

In an embodiment, the patient is orally administered one or more tablets comprising ripretinib. For example, the disclosed methods include a method of treating melanoma in a patient in need thereof, comprising orally administering to the patient one or more tablets comprising ripretinib, e.g., tablets each comprising 50 mg to 100 mg of ripretinib, daily. In some embodiments, the melanoma is a KIT activated melanoma. In some embodiments, the KIT driven melanoma has a baseline mutation selected from the group consisting of a KIT exon 9 mutation, a KIT exon 11 mutation, a KIT exon 13 mutation, a KIT exon 17 mutation, and a KIT exon 18 mutation. In some embodiments, the KIT driven melanoma is caused by overexpression of wildtype KIT. In some embodiments, after at least one 28-day cycle, the patient has a progression-free survival as measured using RECIST 1.1. In some embodiments, the method comprises orally administering to the patient one tablet comprising ripretinib. In some embodiments, the method comprises orally administering to the patient one tablet comprising 50 mg of ripretinib. In some embodiments, the method comprises orally administering to the patient one tablet comprising 50 mg of ripretinib once daily. In some embodiments, the method comprises orally administering to the patient two tablets each comprising 50 mg of ripretinib. In some embodiments, the method comprises orally administering to the patient two tablets each comprising 50 mg of ripretinib once daily. In some embodiments, the method comprises orally administering to the patient three tablets each comprising 50 mg of ripretinib. In some embodiments, the method comprises orally administering to the patient three tablets each comprising 50 mg of ripretinib once daily. In some embodiments, the method comprises orally administering to the patient four tablets each comprising 50 mg of ripretinib. In some embodiments, the method comprises orally administering to the patient four tablets each comprising 50 mg of ripretinib once daily. In some embodiments, the method comprises orally administering to the patient five tablets each comprising 50 mg of ripretinib. In some embodiments, the method comprises orally administering to the patient five tablets each comprising 50 mg of ripretinib once daily. In some embodiments, the method comprises orally administering to the patient six tablets each comprising 50 mg of ripretinib. In some embodiments, the method comprises orally administering to the patient six tablets each comprising 50 mg of ripretinib once daily.

In an embodiment, provided is a method of treating melanoma in a patient in need thereof, comprising orally administering to the patient, on a daily basis, one or more tablets each comprising ripretinib, e.g., tablets each comprising 50 mg to 100 mg of ripretinib, wherein the patient was previously administered at least one tyrosine kinase inhibitor before administration of the ripretinib. In some embodiments, the melanoma is a KIT driven melanoma. In some embodiments, the KIT driven melanoma has a baseline mutation selected from the group consisting of a KIT exon 9 mutation, a KIT exon 11 mutation, a KIT exon 13 mutation, a KIT exon 17 mutation, and a KIT exon 18 mutation. In some embodiments, the KIT driven melanoma is caused by overexpression of wildtype KIT. In some embodiments, after at least one 28-day cycle, the patient has a progression-free survival as measured using RECIST 1.1. In some embodiments, the at least one previously administered tyrosine kinase inhibitor is selected from the group consisting of imatinib, sunitinib, regorafenib, nilotinib, avapritinib, and AZD3229 and pharmaceutically acceptable salts thereof. In some embodiments, the patient has previously been administered two, three, four, or five separate tyrosine kinase inhibitors. In some embodiments, the method comprises orally administering to the patient one tablet comprising ripretinib. In some embodiments, the method comprises orally administering to the patient one tablet comprising 50 mg of ripretinib. In some embodiments, the method comprises orally administering to the patient one tablet comprising 50 mg of ripretinib once daily. In some embodiments, the method comprises orally administering to the patient two tablets each comprising 50 mg of ripretinib. In some embodiments, the method comprises orally administering to the patient, once daily, two tablets each comprising 50 mg of ripretinib. In some embodiments, the method comprises orally administering to the patient three tablets each comprising 50 mg of ripretinib. In some embodiments, the method comprises orally administering to the patient, once daily, three tablets each comprising 50 mg of ripretinib.

In another embodiment, described herein is a method of treating melanoma in a patient in need thereof, comprising administering, e.g., orally administering, to the patient 100 mg to 600 mg of ripretinib daily, and one or more additional therapeutic agents. In some embodiments, the melanoma is a KIT driven melanoma. In some embodiments, the KIT driven melanoma has a baseline mutation selected from the group consisting of a KIT exon 9 mutation, a KIT exon 11 mutation, a KIT exon 13 mutation, a KIT exon 17 mutation, and a KIT exon 18 mutation. In some embodiments, the KIT driven melanoma has a baseline genomic alteration causing the overexpression of wildtype KIT. In some embodiments, after at least one 28-day cycle, the patient has a progression-free survival as measured using RECIST 1.1. In some embodiments, the patient was previously administered at least one tyrosine kinase inhibitor prior to being administered ripretinib. In some embodiments, the at least one previously administered tyrosine kinase inhibitor is selected from the group consisting of imatinib, sunitinib, regorafenib, nilotinib, avapritinib and pharmaceutically acceptable salts thereof. In some embodiments, the patient has previously been administered two, three, four, or five separate tyrosine kinase inhibitors. In some embodiments, the method comprises orally administering to the patient one tablet comprising ripretinib. In some embodiments, the method comprises orally administering to the patient one tablet comprising 50 mg of ripretinib. In some embodiments, the method comprises orally administering to the patient one tablet comprising 50 mg of ripretinib once daily. In some embodiments, the method comprises orally administering to the patient two tablets each comprising 50 mg of ripretinib. In some embodiments, the method comprises orally administering to the patient, once daily, two tablets each comprising 50 mg of ripretinib. In some embodiments, the method comprises orally administering to the patient three tablets each comprising 50 mg of ripretinib. In some embodiments, the method comprises orally administering to the patient, once daily, three tablets each comprising 50 mg of ripretinib.

In another embodiment, described herein is a method of treating melanoma in a patient in need thereof, comprising administering, e.g., orally administering, to the patient 100 mg to 600 mg of ripretinib daily, and one or more additional therapeutic agents. In some embodiments, the melanoma is a KIT driven melanoma. In some embodiments, the KIT driven melanoma has a baseline mutation selected from the group consisting of a KIT exon 9 mutation, a KIT exon 11 mutation, a KIT exon 13 mutation, a KIT exon 17 mutation, and a KIT exon 18 mutation. In some embodiments, the KIT driven melanoma has a baseline genomic alteration causing the overexpression of wildtype KIT. In some embodiments, after at least one 28-day cycle, the patient has a progression-free survival as measured using RECIST 1.1. In some embodiments, the patient was previously administered at least one tyrosine kinase inhibitor prior to being administered ripretinib. In some embodiments, the at least one previously administered tyrosine kinase inhibitor is selected from the group consisting of imatinib, sunitinib, regorafenib, nilotinib, avapritinib and pharmaceutically acceptable salts thereof. In some embodiments, the patient has previously been administered two, three, four, or five separate tyrosine kinase inhibitors. In some embodiments, the method comprises orally administering to the patient one tablet comprising ripretinib. In some embodiments, the method comprises orally administering to the patient one tablet comprising 50 mg of ripretinib. In some embodiments, the method comprises orally administering to the patient one tablet comprising 50 mg of ripretinib once daily. In some embodiments, the method comprises orally administering to the patient two tablets each comprising 50 mg of ripretinib. In some embodiments, the method comprises orally administering to the patient, once daily, two tablets each comprising 50 mg of ripretinib. In some embodiments, the method comprises orally administering to the patient three tablets each comprising 50 mg of ripretinib. In some embodiments, the method comprises orally administering to the patient, once daily, three tablets each comprising 50 mg of ripretinib.

In some embodiments, if the patient suffers from a Grade 3 palmer-plantar erythrodysesthesia syndrome upon administration of ripretinib, the method further comprises a) withholding administration of ripretinib for at least 7 days or until the patient has less than or equal to Grade 1 palmar-plantar erythrodysesthesia syndrome, then administering to the patient 100 mg daily (e.g., 100 mg once daily) ripretinib for at least 28 days.

In some embodiments, if the patient suffers from a Grade 2 palmar-plantar erythrodysesthesia syndrome upon administration of ripretinib, the method further comprises: a) withholding administration of ripretinib until the patient has less than or equal to Grade 1 palmar-plantar erythrodysesthesia syndrome or baseline; b) if the patient recovers from the palmer-plantar erythrodysesthesia syndrome within 7 days of withholding administration, then administering to the patient 150 mg daily ripretinib or c) if the patient has not recovered, then administering to the patient 100 mg daily ripretinib for at least 28 days.

In another embodiment, described herein is a method for achieving at least 5 months of progression free survival as determined by RECIST 1.1 in a patient having melanoma, comprising orally administering to the patient 100, 150 200, or 300 mg of ripretinib daily or twice daily for at least 28 days. In some embodiments, the patient has been administered at least one previous kinase inhibitor. In some embodiments, the patient has been administered at least three previous kinase inhibitors. In some embodiments, the at least one previous kinase inhibitor is imatinib. In some embodiments, comprising orally administering to the patient 100, 150 or 200 mg of ripretinib daily or twice daily for at least 4 months.

In another embodiment, described herein is a method for achieving at least 5 months of progression free survival as determined by RECIST 1.1 in a patient having melanoma, comprising orally administering to the patient 100, 150, or 200 mg of ripretinib daily or twice daily for at least 28 days. In some embodiments, the patient has not been administered a previous kinase inhibitor. In some embodiments, the patient has been administered at least one previous kinase inhibitor. In some embodiments, the patient has been administered at least three previous kinase inhibitors. In some embodiments, the at least one previous kinase inhibitor is imatinib. In some embodiments, comprising orally administering to the patient 100, 150, or 200 mg of ripretinib daily or twice daily for at least 4 months.

In another embodiment, described herein is a method of treating melanoma in a patient in need thereof, wherein the patient is being treated concurrently with a CYP3A4 inhibitor, the method comprising: orally administering to the patient 100 mg or 150 mg of ripretinib, or a pharmaceutically acceptable salt thereof, once or twice daily, and wherein upon administration of the ripretinib and the CYP3A4 inhibitor, provides an increased ripretinib area under the plasma concentration curve (AUC_{0-nf}) of 80% or more in the patient as compared to administration of ripretinib without concurrent treatment of the CYP3A4 inhibitor, and therefore the patient is at higher risk of an adverse event; and monitoring the patient more frequently, as compared to a patient not being treated with a CYP3A4 inhibitor, for the adverse event. In some embodiments, if the patient suffers from a Grade 3 palmar-plantar erythrodysesthesia syndrome adverse event, the method further comprises a) withholding administration of ripretinib for at least 7 days or until the patient has less than or equal to Grade 1 palmar-plantar erythrodysesthesia syndrome, then administering to the patient 100 mg daily ripretinib for at least 28 days. In some embodiments, if the patient suffers from Grade 2 palmar-plantar erythrodysesthesia syndrome upon administration of the ripretinib, the method further comprises: a) withholding administration of ripretinib until the patient has less than or equal to Grade 1 palmar-plantar erythrodysesthesia syndrome or baseline; b) if the patient recovers from the palmer-plantar erythrodysesthesia syndrome within 7 days of withholding administration, then administering to the patient 150 mg daily ripretinib or c) if the patient has not recovered, then administering to the patient 100 mg daily ripretinib for at least 28 days. In some embodiments, the CYP3A4 inhibitor is selected from the group consisting of itraconazole, ketoconazole, clarithromycin, and indinavir. In some embodiments, the CYP3A4 inhibitor is itraconazole. In some embodiments, the patient has previously been administered one or more tyrosine kinase inhibitors, each selected from the group consisting of imatinib, sunitinib, regorafenib, nilotinib, avapritinib, and pharmaceutically acceptable salts thereof.

In another embodiment, described herein is a method of treating melanoma in a patient in need thereof, wherein the patient is being treated concurrently with a proton pump inhibitor, the method comprising: orally administering to the patient 100 mg or 150 mg of ripretinib, or a pharmaceutically acceptable salt thereof, once or twice daily, and wherein administration of the ripretinib and proton pump inhibitor to the patient provides no clinically significant difference in the plasma exposure of ripretinib in the patient as compared to administration of ripretinib without concurrent treatment of the proton pump inhibitor. In some embodiments, the proton pump inhibitor is selected from the group consisting of pantoprazole, omeprazole, lansoprazole, rabeprazole, esomeprazole, and dexlansoprazole. In some embodiments, the proton pump inhibitor is pantoprazole. In some embodiments, the patient is being treated concurrently with 40 mg of the proton pump inhibitor once daily.

In another embodiment, described herein is a method of treating melanoma in a patient in need thereof, the method comprising orally administering to the patient 100 mg or 150 mg of ripretinib, or a pharmaceutically acceptable salt thereof, once or twice daily, wherein the ripretinib is administered to the patient with food or without food. In some embodiments, the food comprises a high-fat meal (e.g., a high-fat meal described herein).

In some embodiments, the therapeutic efficacy of ripretinib is determined by the progression-free survival of the patient after independent radiologic review using Response Evaluation Criteria in Solid Tumors (RECIST) 1.1. In some embodiments, the therapeutic efficacy of ripretinib is determined by the Objective Response Rate (ORR), Time to Tumor Progression (TTP) or Overall Survival (OS) of the patient after independent radiologic review using RECIST 1.1. In some embodiments, the therapeutic efficacy of ripretinib is determined by the progression-free survival of the patient based on investigator assessment.

After at least one month, two months, e.g., 28 days or more of treatment with ripretinib, the patient may have a progression-free survival as measured using RECIST 1.1.

### Dose Modifications

Dose modifications may be made in the methods of administering ripretinib described herein as a result of adverse events experienced by the patient. In some embodiments, the dose modification is a dose interruption. In some embodiments, the dose modification is a permanent discontinuation in dosing. In some embodiments, the dose modification is a dose reduction. In some embodiments, the dose of ripretinib administered to the patient is reduced from 150 mg once daily, e.g., three tablets each comprising 50 mg of ripretinib, to 100 mg once daily, e.g., two tablets each comprising 50 mg of ripretinib. In some embodiments, the dose of ripretinib administered to the patient is reduced from 150 mg once daily, e.g., three tablets each comprising 50 mg of ripretinib, to 50 mg once daily, e.g., one tablet comprising 50 mg of ripretinib. In some embodiments, the adverse reaction is selected from the group consisting of a hand-foot skin reaction (e.g., palmar-plantar erythrodysesthesia syndrome), hypertension, arthralgia, and myalgia.

In some embodiments, the adverse event is graded in accordance with the National Cancer Institute Common Terminology Criteria for Adverse Events, version 4.03 (e.g., baseline, Grade 1, Grade 2, Grade 3, or Grade 4). In some embodiments, the dose modification is a dose interruption (e.g., a dose interruption of at least 7 days) as a result of a Grade 2 adverse event. In some embodiments, dosing resumes at the same dose level before the dose interruption if the adverse event is lowered to Grade 1 or baseline within a first time period (e.g., within 7 days). In some embodiments, dosing resumes at a reduced dose level before the dose interruption if the adverse event is lowered to Grade 1 or baseline after a first time period (e.g., after 7 days). In some embodiments, the reduced dose level is re-escalated to the dose level prior to the dose interruption if the adverse event is lowered to Grade 1 or baseline after a first time period but is maintained as a Grade 1 or baseline adverse event after a second time period (e.g., after 28 days). In some embodiments, the dose modification is a dose interruption (e.g., a dose interruption of at least 7 days up to a maximum of 28 days) as a result of a Grade 3 adverse event. In some embodiments, dosing is continued at a reduced level after the dose interruption. In some embodiments, the dose modification is a permanent discontinuation in dosing as a result of a Grade 4 adverse event (e.g., Grade 4 hypertension).

A patient can be administered an additional treatment in response to an adverse event or to prevent an adverse event from occurring. In some embodiments, a patient suffering from an adverse dermatologic reaction, e.g., a hand-foot skin reaction, e.g., palmar-plantar erythrodysesthesia syndrome, is administered a topical composition (e.g., an emollient) to treat the adverse dermatologic reaction. In some embodiments, the patient is administered the topical composition (e.g., an emollient) based on the severity of the adverse dermatologic reaction, e.g., a Grade 2, Grade 3 adverse dermatologic reaction, e.g., a Grade 1, Grade 2, or Grade 3 hand-foot skin reaction, e.g., a Grade 1, Grade 2 or Grade 3 palmar-plantar erythrodysesthesia syndrome. In some embodiments, the topical composition (e.g., an emollient) is administered to the patient during a dose interruption of ripretinib. In some embodiments, the topical composition (e.g., an emollient) is administered to the patient contemporaneously with a dose of ripretinib, e.g., a reduced dose of ripretinib.

A patient can also be administered an additional treatment prior to, or during administration of ripretinib in accordance with the methods described herein to prevent or ameliorate an adverse event. In some embodiments, the patient is administered a topical composition (e.g., an emollient) before and/or during ripretinib administration to prevent or ameliorate the onset of an adverse dermatologic reaction, e.g., a hand-foot skin reaction, e.g., palmar-plantar erythrodysesthesia syndrome.

### Combination Therapy

The present disclosure describes, in an embodiment, combination therapies that involve the administration of ripretinib or a composition comprising ripretinib, and one or more therapeutic agents. The combination therapies described herein can be used by themselves, or in further combination with one or more additional therapeutic agents (e.g., one or more additional therapeutic agents described below). For example, the compound of Formula (I) or a composition comprising an amorphous form compound of Formula (I) can be administered together with a cancer targeted therapeutic agent, a cancer-targeted biological, an immune checkpoint inhibitor, or a chemotherapeutic agent. The therapeutic agents can be administered together with or sequentially with another therapeutic agent described herein in a combination therapy.

Combination therapy can be achieved by administering two or more therapeutic agents, each of which is formulated and administered separately. Alternatively, combination therapy can be achieved by administering two or more therapeutic agents in a single formulation.

Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X-Y-X, X-X-Y, Y-X-Y, Y-Y-X, X-X-Y-Y, etc.

In some embodiments, the additional therapeutic agent that may be administered according to the present disclosure include, but are not limited to, cytotoxic agents, cisplatin, doxorubicin, etoposide, irinotecan, topotecan, paclitaxel, docetaxel, the epothilones, tamoxifen, 5-fluorouracil, methotrexate, temozolomide, cyclophosphamide, lonafarnib, tipifarnib, 4-((5-((4-(3-chlorophenyl)-3-oxopiperazin-1-yl)methyl)-1H-imidazol-1-yl)methyl)benzonitrile hydrochloride, (R)-1-((1H-imidazol-5-yl)methyl)-3-benzyl-4-(thiophen-2-ylsulfonyl)-2,3,4,5-tetrahydro-1H-benzo diazepine-7-carbonitrile, cetuximab, imatinib, interferon alfa-2b, pegylated interferon alfa-2b, aromatase combinations, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, leucovorin, oxaliplatin, pentostatine, vinblastine, vincristine, vindesine, bleomycin, dactinomycin, daunorubicin, epirubicin, idarubicin, mithramycin, deoxycoformycin, mitomycin-C, L-asparaginase, teniposide 17α-ethinyl estradiol, diethylstilbestrol, testosterone, prednisone, fluoxymesterone, dromostanolone propionate, testolactone, megestrol acetate, methylprednisolone, methyltestosterone, prednisolone, triamcinolone, chlorotrianisene, 17α-hydroxyprogesterone, aminoglutethimide, estramustine, medroxyprogesterone acetate, leuprolide acetate, flutamide, toremifene citrate, goserelin acetate, carboplatin, hydroxyurea, amsacrine, procarbazine, mitotane, mitoxantrone, levamisole, vinorelbine, anastrazole, letrozole, capecitabine, raloxifene, droloxifene, hexamethylmelamine, bevacizumab, trastuzumab, tositumomab, bortezomib, ibritumomab tiuxetan, arsenic trioxide, porfimer sodium, cetuximab, thioTEPA, altretamine, fulvestrant, exemestane, rituximab, alemtuzumab, dexamethasone, bicalutamide, chlorambucil, and valrubicin.

In some embodiments, the additional therapeutic agent that can be administered may include, without limitation, a DNA-damaging (or DNA-alkylating) agent, an AKT inhibitor, alkylating agent, all-trans retinoic acid, antiandrogen, azacitidine, BCL2 inhibitor, BCL-XL inhibitor, BCR-ABL inhibitor, BTK inhibitor, BTK/LCK/LYN inhibitor, CDK1/2/4/6/7/9 inhibitor, CDK4/6 inhibitor, CDK9 inhibitor, CBP/p300 inhibitor, EGFR inhibitor (e.g., afatinib, neratinib), endothelin receptor antagonist, RAF inhibitor, MEK (mitogen-activated protein kinase kinase) inhibitor, ERK inhibitor, farnesyltransferase inhibitor, FLT3 inhibitor, glucocorticoid receptor agonist, HDM2 inhibitor, histone deacetylase inhibitor, IKKβ inhibitor, immunomodulatory drug (IMiD), ingenol, ITK inhibitor, JAK1/JAK2/JAK3/TYK2 inhibitor, MTOR inhibitor, PI3 kinase inhibitor, dual PI3 kinase/MTOR inhibitor, proteasome inhibitor, protein kinase C agonist, SUV39H1 inhibitor, TRAIL, VEGFR2 inhibitor, Wnt/β-catenin signaling inhibitor, decitabine, and anti-CD20 monoclonal antibody.

In some embodiments, the additional therapeutic agent is an immunomodulatory agent selected from the group consisting of CTLA4 inhibitors such as, but not limited to ipilimumab and tremelimumab; PD1 inhibitors such as, but not limited to pembrolizumab, and nivolumab; PDL1 inhibitors such as, but not limited to atezolizumab (formerly MPDL3280A), durvalumab (formerly MEDI4736), avelumab, PDR001; 4 1BB or 4 1BB ligand inhibitors such as, but not limited to urelumab and PF-05082566; OX40 ligand agonists such as, but not limited to MEDI6469; GITR agents such as, but not limited to TRX518; CD27 inhibitors such as, but not limited to varlilumab; TNFRSF25 or TL1A inhibitors; CD40 agonists such as, but not limited to CP-870893; HVEM or LIGHT or LTA or BTLA or CD160 inhibitors; LAG3 inhibitors such as, but not limited to BMS-986016; TIM3 inhibitors; Siglecs inhibitors; ICOS or ICOS ligand agonists; B7 H3 inhibitors such as, but not limited to MGA271; B7 H4 inhibitors; VISTA inhibitors; HHLA2 or TMIGD2 inhibitors; inhibitors of Butyrophilins, including BTNL2 inhibitors; CD244 or CD48 inhibitors; inhibitors of TIGIT and PVR family members; KIRs inhibitors such as, but not limited to lirilumab; inhibitors of ILTs and LIRs; NKG2D and NKG2A inhibitors such as, but not limited to IPH2201; inhibitors of MICA and MICB; CD244 inhibitors; CSF1R inhibitors such as, but not limited to vimseltinib, emactuzumab, cabiralizumab, pexidartinib, ARRY382, BLZ945; IDO inhibitors such as, but not limited to INCB024360; thalidomide, lenalidomide, TGFβ inhibitors such as, but not limited to galunisertib; adenosine or CD39 or CD73 inhibitors; CXCR4 or CXCL12 inhibitors such as, but not limited to ulocuplumab and (3S,6S,9S,12R,17R,20S,23S,26S,29S,34aS)-N-((S)-1-amino-5-guanidino-1-oxopentan-2-yl)-26,29-bis(4-aminobutyl)-17-((S)-2-((S)-2-((S)-2-(4-fluorobenzamido)-5-guanidinopentanamido)-5-guanidinopentanamido)-3-(naphthalen-2-yl)propanamido)-6-(3-guanidinopropyl)-3,20-bis(4-hydroxybenzyl)-1,4,7,10,18,21,24,27,30-nonaoxo-9,23-bis(3-ureidopropyl)triacontahydro-1H,16H-pyrrolo[2,1-p][1,2]dithia[5,8,11,14,17,20,23,26,29]nonaazacyclodotriacontine-12-carboxamide BKT140; phosphatidylserine inhibitors such as, but not limited to bavituximab; SIRPA or CD47 inhibitors such as, but not limited to CC-90002; VEGF inhibitors such as, but not limited to bevacizumab; and neuropilin inhibitors such as, but not limited to MNRP1685A.

In some embodiments, the additional therapeutic agent is a chemotherapeutic agent selected from the group consisting of chemotherapeutic agents including but not limited to anti-tubulin agents (paclitaxel, paclitaxel protein-bound particles for injectable suspension such as nab-paclitaxel, eribulin, docetaxel, ixabepilone, taxiterem, vincristine, or vinorelbine), vinorelbine, LHRH antagonists including but not limited to leuprolide, goserelin, triptorelin, or histrelin, anti-androgen agents including but not limited to abiraterone, flutamide, bicalutamide, nilutamide, cyproterone acetate, enzalutamide, and apalutamide, anti-estrogen agents including but not limited to tamoxifen, fulvestrant, anastrozole, letrozole, and exemestane, DNA-alkylating agents (including cisplatin, carboplatin, oxaliplatin, cyclophosphamide, ifosfamide, and temozolomide), DNA intercalating agents (including doxorubicin, pegylated liposomal doxorubicin, daunorubicin, idarubicin, and epirubicin), 5-fluorouracil, capecitabine, cytarabine, decitabine, 5-aza cytadine, gemcitabine, bortezomib, and carfilzomib, and methotrexate.

In some embodiments, the additional therapeutic agent is selected from the group consisting of paclitaxel, paclitaxel protein-bound particles for injectable suspension, eribulin, docetaxel, ixabepilone, vincristine, vinorelbine, cisplatin, carboplatin, oxaliplatin, cyclophosphamide, ifosfamide, temozolomide, doxorubicin, pegylated liposomal doxorubicin, daunorubicin, idarubicin, epirubicin, 5-fluorouracil, capecitabine, cytarabine, decitabine, 5-azacytidine, gemcitabine, methotrexate, erlotinib, gefitinib, lapatinib, everolimus, temsirolimus, LY2835219, LEE011, PD 0332991, crizotinib, cabozantinib, sunitinib, pazopanib, sorafenib, regorafenib, axitinib, dasatinib, imatinib, nilotinib, vemurafenib, dabrafenib, trametinib, idelasib, quizartinib, tamoxifen, fulvestrant, anastrozole, letrozole, exemestane, abiraterone acetate, enzalutamide, nilutamide, bicalutamide, flutamide, cyproterone acetate, prednisone, dexamethasone, irinotecan, camptothecin, topotecan, etoposide, etoposide phosphate, mitoxantrone, vorinostat, romidepsin, panobinostat, valproic acid, belinostat, DZNep 5-aza-2′-deoxycytidine, bortezomib, carfilzomib, thalidomide, lenalidomide, pomalidomide, trastuzumab, pertuzumab, cetuximab, panitumumab, ipilimumab, lambrolizumab, nivolumab, MPDL3280A, bevacizumab, aflibercept, brentuximab vedotin, ado-trastuzumab emtansine, radiotherapy, and sipuleucel T.

In some embodiments, the additional therapeutic agent is a kinase inhibitor selected from the group consisting of erlotinib, gefitinib, lapatinib, everolimus, temsirolimus, LY2835219, LEE011, PD 0332991, crizotinib, cabozantinib, sunitinib, pazopanib, sorafenib, regorafenib, axitinib, dasatinib, imatinib, nilotinib, vemurafenib, dabrafenib, trametinib, idelalisib, and quizartinib.

In some embodiments, the additional therapeutic agent is an anti-PD1 therapeutic. Examples of anti-PD1 therapeutics that may be administered in combination with the compound of Formula (I) or pharmaceutically acceptable salt thereof or a composition comprising the compound of Formula (I) or pharmaceutically acceptable salt thereof described herein include, but are not limited to, nivolumab, pidilizumab, cemiplimab, tislelizumab, AMP-224, AMP-514, and pembrolizumab.

In some embodiments, the additional therapeutic agent is selected from the group consisting of immunomodulatory agents including but not limited to anti-PD-L1 therapeutics including atezolizumab, durvalumab, BMS-936559, and avelumab, anti-TIM3 therapeutics including TSR-022 and MBG453, anti-LAG3 therapeutics including relatlimab, LAG525, and TSR-033, CD40 agonist therapeutics including SGN-40, CP-870,893 and RO7009789, anti-CD47 therapeutics including Hu5F9-G4, anti-CD20 therapeutics, anti-CD38 therapeutics, and other immunomodulatory therapeutics including thalidomide, lenalidomide, pomalidomide, prednisone, and dexamethasone. In some embodiments, the additional therapeutic agent is avelumab.

In some embodiments, the additional therapeutic agent is selected from the group consisting of targeted therapeutics including kinase inhibitors erlotinib, gefitinib, lapatinib, everolimus, temsirolimus, abemaciclib, LEE011, palbociclib, crizotinib, cabozantinib, sunitinib, pazopanib, sorafenib, regorafenib, axitinib, dasatinib, imatinib, nilotinib, vemurafenib, dabrafenib, trametinib, cobimetinib, binimetinib, idelalisib, quizartinib, avapritinib, BLU-667, BLU-263, Loxo 292, larotrectinib, and quizartinib, anti-estrogen agents including but not limited to tamoxifen, fulvestrant, anastrozole, letrozole, and exemestane, anti-androgen agents including but not limited to abiraterone acetate, enzalutamide, nilutamide, bicalutamide, flutamide, cyproterone acetate, steroid agents including but not limited to prednisone and dexamethasone, PARP inhibitors including but not limited to niraparib, olaparib, and rucaparib, topoisomerase I inhibitors including but not limited to irinotecan, camptothecin, and topotecan, topoisomerase II inhibitors including but not limited to etoposide, etoposide phosphate, and mitoxantrone, Histone Deacetylase (HDAC) inhibitors including but not limited to vorinostat, romidepsin, panobinostat, valproic acid, and belinostat, DNA methylation inhibitors including but not limited to DZNep and 5-aza-2′-deoxycytidine, proteasome inhibitors including but not limited to bortezomib and carfilzomib, thalidomide, lenalidomide, pomalidomide, biological agents including but not limited to trastuzumab, ado-trastuzumab, pertuzumab, cetuximab, panitumumab, ipilimumab, tremelimumab, vaccines including but not limited to sipuleucel-T, and radiotherapy. In some embodiments, the additional therapeutic agent is selected from the group consisting of KIT inhibitors. In some embodiments, the additional therapeutic agent is selected from the group consisting of imatinib, sunitinib, regorafenib, nilotinib, and avapritinib. In some embodiments, the additional therapeutic agent is selected from the group consisting of imatinib, sunitinib, regorafenib, nilotinib, avapritinib, and AZD3229.

In some embodiments, the additional therapeutic agent is selected from the group consisting of an inhibitor of the TIE2 immunokinase including rebastinib or ARRY-614.

In some embodiments, the additional therapeutic agent is selected from the group consisting of an inhibitor of the TIE2 immunokinase including rebastinib or ARRY-614, and an anti-PD1 therapeutic.

In some embodiments, the additional therapeutic agent is selected from the group consisting of anti-angiogenic agents including AMG386, bevacizumab and aflibercept, and antibody-drug-conjugates (ADCs) including brentuximab vedotin, trastuzumab emtansine, and ADCs containing a payload such as a derivative of camptothecin, a pyrrolobenzodiazepine dimer (PBD), an indolinobenzodiazepine dimer (IGN), DM1, DM4, MMAE, or MMAF.

In some embodiments, the additional therapeutic agent is selected from a luteinizing hormone-releasing hormone (LHRH) analog, including goserelin and leuprolide.

In some embodiments, the additional therapeutic agent is selected from the group consisting of selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, pemetrexed, erlotinib, dasatinib, nilotinib, dasatinib, panitumumab, amrubicin, oregovomab, Lep-etu, nolatrexed, azd2171, batabulin, of atumtunab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR1 KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)-ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258); 3-[5-(methylsulfonylpiperadinemethyl)-indolylj-quinolone, vatalanib, AG-013736, AVE-0005, the acetate salt of [D-Ser(Bu t) 6, Azgly 10](pyro-Glu-His-Trp-Ser-Tyr-D-Ser(Bu t)-Leu-Arg-Pro-Azgly-NH2 acetate [C59H84N18Oi4-(C2H402)x where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatinib, canertinib, ABX-EGF antibody, erbitux, EKB-569, PKI-166, GW-572016, Ionafarnib, BMS-214662, tipifarnib; amifostine, NVP-LAQ824, suberoyl anilide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deoxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxifene, spironolactone, finasteride, cimetidine, trastuzumab, denileukin diftitox, gefitinib, bortezomib, irinotecan, topotecan, doxorubicin, docetaxel, vinorelbine, bevacizumab (monoclonal antibody) and erbitux, cremophor-free paclitaxel, epothilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zoledronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritumomab tiuxetan, androgens, decitabine, hexamethylmelamine, bexarotene, tositumomab, arsenic trioxide, cortisone, etidronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, ipilimumab, vemurafenib, and mixtures thereof. In some embodiments, the additional therapeutic agent is selected from the group consisting of imatinib, sunitinib, regorafenib, nilotinib, and avapritinib.

In some embodiments, the additional therapeutic agent is an HSP90 inhibitor (e.g., AT13387). In some embodiments, the additional therapeutic agent is cyclophosphamide. In some embodiments, the additional therapeutic agent is an AKT inhibitor (e.g., perifosine). In some embodiments, the additional therapeutic agent is a BCR-ABL inhibitor (e.g., nilotinib). In some embodiments, the additional therapeutic agent is an mTOR inhibitor (e.g., RAD001). In some embodiments, the additional therapeutic agent is an FGFR inhibitor (e.g., erdafitinib, KO947, or BGJ398). In some embodiments, the additional therapeutic agent is an anti-PDL1 therapeutic. In some embodiments, the additional therapeutic agent is a Bcl2 inhibitor (e.g., venetoclax). In some embodiments, the additional therapeutic agent is an autophagy inhibitor (e.g., hydroxychloroquine). In some embodiments, the additional therapeutic agent is a MET inhibitor.

In some embodiments, the additional therapeutic agent is an inhibitor of the MAPK pathway. In some embodiments, the additional therapeutic agent is a RAF inhibitor. In some embodiments, the additional therapeutic agent is a RAF inhibitor selected from the group consisting of belvarafenib, LXH-254, lifirafenib (BGB-283), AZ-268, TAK-632, sorafenib, LY3009120, vemurafenib, dabrafenib, encorafenib, and PLX8394. In some embodiments, the additional therapeutic agent is a MEK inhibitor. In some embodiments, the additional therapeutic agent is a MEK inhibitor selected from the group consisting of binimetinib, cobimetinib, selumetinib, trametinib, pimasertib, and PD-0325901. In some embodiments, the additional therapeutic agent is an ERK inhibitor. In some embodiments, the additional therapeutic agent is an ERK inhibitor selected from the group consisting of GDC-0994, KO-947, LY-3214996, Vtx-11e, SCH-772984. MK-8253, and ulixertinib.

In some embodiments, the additional therapeutic agent is an inhibitor of the PI3K pathway. In some embodiments, the additional therapeutic agent is a p110 inhibitor. In some embodiments, the additional therapeutic agent is a p110 inhibitor selected from the group consisting of alpelisib, copanlisib, duvelisib, idelalisib, GDC-0077, and taselisib. In some embodiments, the additional therapeutic agent is an AKT inhibitor. In some embodiments, the additional therapeutic agent is an AKT inhibitor and is ipatasertib. In some embodiments, the additional therapeutic agent is an mTOR inhibitor. In some embodiments, the additional therapeutic agent is an mTOR inhibitor selected from the group consisting of everolimus and temsirolimus.

### Examples

### Example 1. A Phase 1 Study of Treating Melanoma with Ripretinib

This is a Phase 1 study to evaluate the efficacy of ripretinib in treating patients with melanoma. An efficacy cohort of 26 patients were enrolled. Each patient was administered 150 mg of ripretinib daily in repeated 28-day cycles until disease progression, unacceptable toxicity, or consent withdrawal. Patients who had disease progression at ripretinib 150 mg QD were allowed to escalate to 150 mg twice daily (BID) after the completion of Cycle 2. The efficacy endpoints includes Objective Response Rate (ORR), Disease Control Rate (DCR), Duration of Response (DOR) Time to Best Response (TBR), and progression-free survival (PFS) as determined by radiologic review using Response Evaluation Criteria in Solid Tumors (RECIST 1.1).

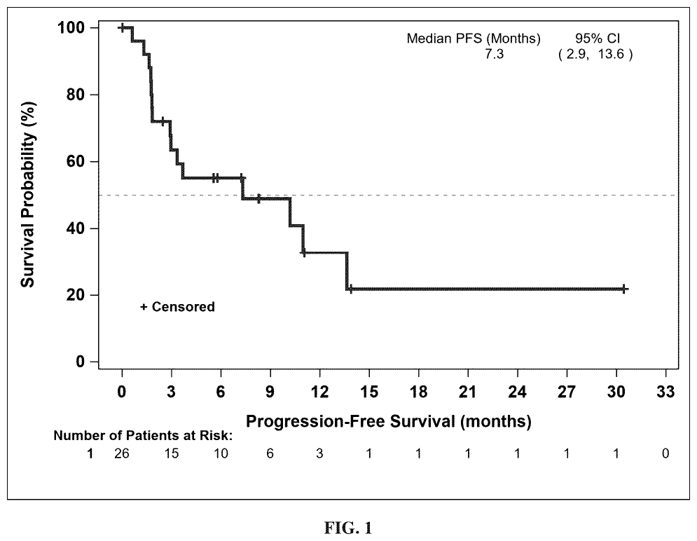

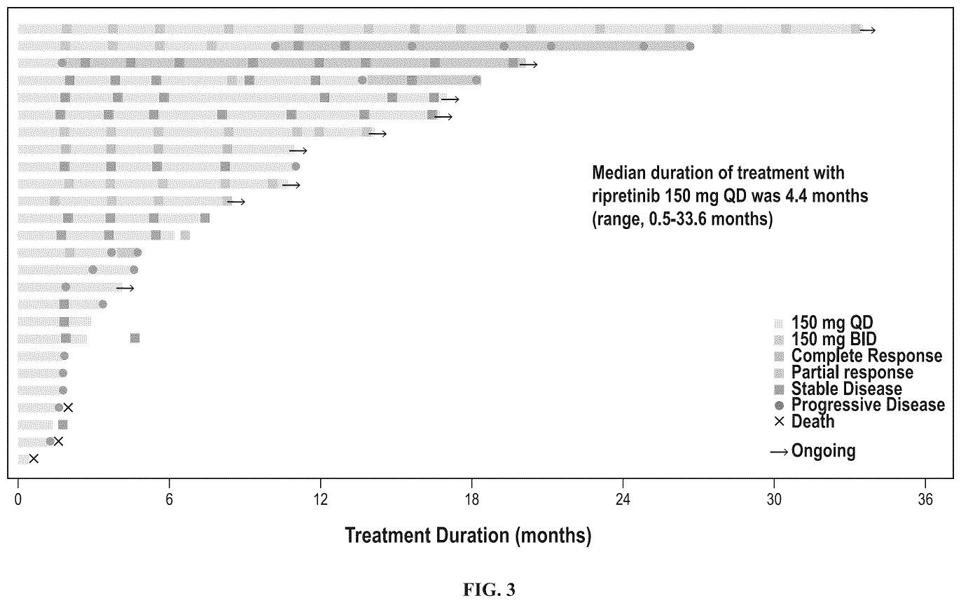

Results. The median duration of treatment with ripretinib at 150 mg QD was 4.4 months (range, 0.5-33.6 months). FIG. 3 shows duration of treatment with ripretinib in individual patients. Ripretinib demonstrated a median PFS of 7.3 months. A plot of survival probability with respect to PFS for patients on ripretinib is shown in FIG. 1. For the secondary endpoint of objective response rate (ORR), as determined by radiologic review using RECIST version 1.1, ripretinib demonstrated a confirmed ORR of 23.1% (6 out of 26 members). Further, ripretinib demonstrated an confirmed and unconfirmed ORR of 30.8%. For the secondary endpoint of duration of response (DOR), ripretinib demonstrated a median DOR (mDOR) of 9.1 months. Ripretinib dose was escalated to 150 mg BID after progressive disease (PD) on 150 mg QD in 4 (15%) of patients.

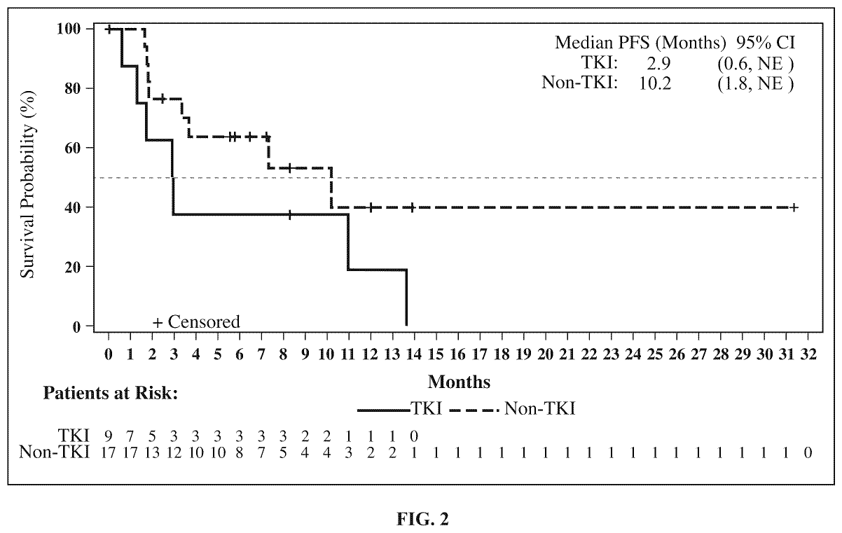

Prior tyrosine kinase inhibitor (TKI) treatment analysis. The efficacy of ripretinib among efficacy cohort members who received prior TKI treatment and the efficacy among members who did not receive prior TKI treatment were evaluated. 17 of the 25 members of the cohort received no prior TKI and, among these members, the mPFS was determined to be 10.2 months and the ORR was determined to be 29.4% (5 out of 17 members). 8 of the 25 members received TKI treatment prior to receiving ripretinib and, among these members, the mPFS was determined to be 2.9 months and the ORR was determined to be 11% (1 out of 9 members). Plots illustrating mPFS among cohort members who did not receive prior TKI treatment (“Non-TKI”) and who did receive prior TKI treatment (“TKI”) are provided in FIG. 2.

Furthermore, overall response results as determined by investigator assessment among the cohort population are provided in Table 1 below.

TABLE 1

| Parameters | Statistics | Total | |
|---|---|---|---|
| Best Overall Response | | | |
| Confirmed Complete Response | n (%) | 1 | (3.8) |
| Confirmed Partial Response | n (%) | 5 | (19.2) |
| Stable Disease (≥6 weeks duration) | n (%) | 11 | (42.3) |
| Progressive Disease | n (%) | 8 | (31) |
| Not Evaluable | n (%) | 0 | |
| No Response Assessment | n (%) | 1 | (4) |
| Objective Response Rate | n (%) | 6 | (23.1) |
| | 95% CI | 9.0, 43.6 | |
| Duration of Response (Months) | n | 6 | |
| | Mean (SD) | 10.5 | (9.01) |
| | Median (range) | 9.1 | (6.9-31.3) |
| | Min, Max | 4.1, 28.6 | |
| Time to Response (Months) | Median (range) | 1.9 | (1.4-2.0) |

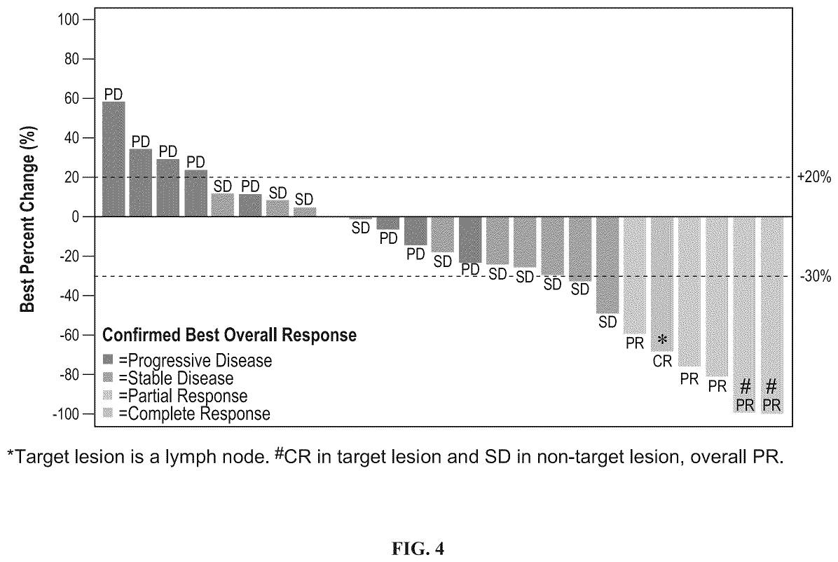

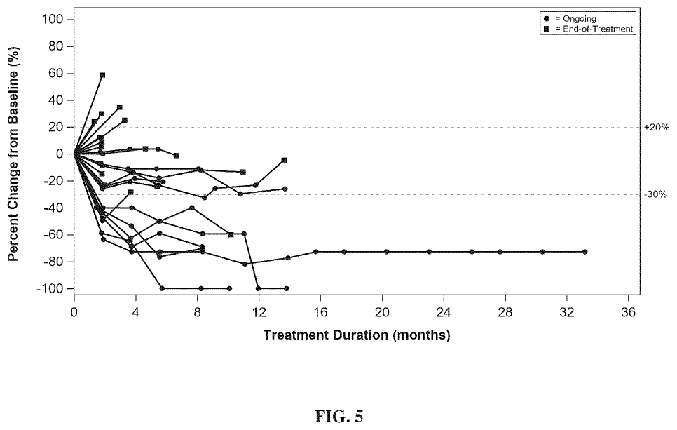

Further, FIG. 4 depicts best overall response in individual patients and their respective percentage changes of target lesion from baseline after ripretinib treatment. FIG. 5 depicts a spider plot of changes in target lesion in individual patients with respect to treatment duration.

Mutational Analysis. An analysis of the efficacy of ripretinib based on baseline mutations of melanoma among the efficacy cohort was performed. Table 2 shows exemplary overall response results with respect to baseline exon mutations observed. L576P (Exon 11), K642E (Exon 13) and N822K/Y (Exon 17) were determined to be the most prevalent baseline mutations in this cohort.

TABLE 2

| | Parameters | | | | | | | | | | |
|---|---|---|---|---|---|---|---|---|---|---|---|
| | | | | KIT | | KIT | | KIT | | KIT | KIT Exon |
| Best Overall | | Total | | Exon 11 | | Exon 13 | | Exon 17 | | Exon 18 | Amplification |
| Response | Statistics | (N = 26) | | (N = 9) | | (N = 4) | | (N = 11) | | (N = 1) | (N = 1) |
| Confirmed Complete | n (%) | 1 | (3.8) | 0 | | 0 | | 1 | (9.1) | 0 | 0 |
| Response | | | | | | | | | | | |
| Confirmed Partial | n (%) | 5 | (19.2) | 4 | (44.4) | 0 | | 1 | (9.1) | 0 | 0 |
| Response | | | | | | | | | | | |
| Stable Disease | n (%) | 11 | (42.3) | 3 | (33.3) | 2 | (50.0) | 5 | (45.5) | 0 | 1 (100.0) |
| (≥6 wks) | | | | | | | | | | | |
| Progressive Disease | n (%) | 8 | (31) | 1 | (11.1) | 2 | (50.0) | 4 | (36) | 1 (100.0) | 0 |

| Not Evaluable | n (%) | 0 | | 0 | | 0 | 0 | 0 | 0 |
|---|---|---|---|---|---|---|---|---|---|
| No Response | n (%) | 1 | (4) | 1 | (11.1) | 0 | 0 | 0 | 0 |

Assessment

Further, the confirmed ORR in Exon 11 and Exon 17 patients was 44% and 18%, respectively. The median duration of response in Exon 11 and Exon 17 patients was 10.5 months (range: 8.3 months-31.3 months) and 8.1 months (range: 6.9 months-9.2 months), respectively. The median PFS in Exon 11 and Exon 17 patients was 10.2 months (95% CI: 0.6 months-not evaluated) and 13.6 months (95% CI: 1.8 months-not evaluated), respectively.

Additionally, the median time to confirmed response (range) in the Exon 11 and Exon 17 patients was 1.9 months (1.8-2.0 months) and 1.7 months (1.4-1.9 months), respectively.

The above data show that melanoma patients in this Phase 1 trial respond well to ripretinib treatment at the mutational level with regards to KIT inhibition.

### Example 2. Studies of Ripretinib and Compound A with Strong CYP3A Inhibitors

Coadministration of 150 mg QD ripretinib with a strong CYP3A inhibitor increased the exposure of ripretinib and its active metabolite (Compound A), which may increase the risk of adverse reactions. Coadministration of ripretinib with itraconazole (a strong CYP3A inhibitor and also a P-gp inhibitor) increased ripretinib Cₘₐₓ by 36% and AUC_{0-inf} by 99% and also increased Compound A AUC_{0-inf} by 99% with no change in its Cₘₐₓ.

### Example 3. Studies of Ripretinib with a Proton-Pump Inhibitor

The effect of a proton-pump inhibitor on the exposure of ripretinib was evaluated. No clinically significant differences in the plasma exposure to ripretinib and Compound A were observed when ripretinib was coadministered with pantoprazole, a proton-pump inhibitor. Although ripretinib has pH-dependent solubility, concomitant administration of 40 mg QD pantoprazole with 150 mg QD ripretinib did not affect ripretinib exposure.

### Example 4. Studies of Food Effect on Ripretinib and Compound A Exposure

The effect of a high-fat breakfast on ripretinib and Compound A exposure was evaluated. A high fat meal consisted of approximately 150, 250, and 500-600 calories from protein, carbohydrate, and fat, respectively. Following administration of ripretinib with a high-fat meal at a 150 mg dose, ripretinib AUC₀₋₂₄ₕ and Cₘₐₓ were higher by 30% and 22%, respectively. For the metabolite Compound A, AUC₀₋₂₄ₕ and Cₘₐₓ were higher by 47% and 66%, respectively. The food effect is not considered to be clinically significant based on exposure-response analysis. Therefore, ripretinib may be taken with or without food at approximately same time each day.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A method of treating a KIT driven melanoma in a patient in need thereof, comprising orally administering to the patient 100 mg to 600 mg of ripretinib daily.

2. The method of claim 1, comprising orally administering to the patient 100 mg to 300 mg of ripretinib daily.

3. The method of claim 1 or 2, comprising orally administering to the patient 150 mg of ripretinib daily.

4. A method of treating a KIT driven melanoma in a patient in need thereof, comprising orally administering to the patient one or more tablets comprising 100 mg to 600 mg ripretinib daily.

5. The method of claim 4, comprising orally administering to the patient three tablets each comprising 50 mg of ripretinib once daily.

6. The method of claim 4, comprising orally administering to the patient two tablets each comprising 50 mg of ripretinib once daily.

7. The method of claim 4, comprising orally administering to the patient one tablet comprising 50 mg of ripretinib twice daily.

8. A method of treating melanoma in a patient in need thereof, comprising orally administering to the patient 100 mg to 600 mg of ripretinib daily, wherein the patient has not been previously administered one or more tyrosine kinase inhibitors before administration of the ripretinib.

9. A method of treating melanoma in a patient in need thereof, comprising orally administering to the patient 100 mg to 600 mg of ripretinib daily, wherein the patient was previously administered at least one tyrosine kinase inhibitor before administration of the ripretinib.

10. The method of claim 8 or 9, comprising orally administering to the patient 100 mg to 300 mg of ripretinib daily.

11. The method of any one of claims 8-10, comprising orally administering to the patient 150 mg of ripretinib daily.

12. The method of any one of claims 8-11, wherein the patient was previously administered at least two, three, four, or five tyrosine kinase inhibitors before administration of the ripretinib.

13. The method of any one of claims 8-12, wherein the at least one previously administered tyrosine kinase inhibitor is selected from the group consisting of imatinib, sunitinib, regorafenib, lapatinib, dasatinib, crizotinib, gefitinib, erlotinib, vatalanib, crenolanib, and pharmaceutically acceptable salts thereof.

14. A method of treating melanoma in a patient in need thereof, comprising orally administering to the patient, on a daily basis, one or more tablets each comprising ripretinib, wherein the patient was previously administered at least one tyrosine kinase inhibitor before administration of the ripretinib.

15. The method of claim 14, comprising administering, once daily, three tablets each comprising 50 mg of ripretinib.

16. The method of claim 14 or 15, wherein the at least one previously administered tyrosine kinase inhibitor is selected from the group consisting of imatinib, sunitinib, regorafenib, lapatinib, dasatinib, crizotinib gefitinib, erlotinib, vatalanib, crenolanib, and pharmaceutically acceptable salts thereof.

17. The method of any one of claims 1-16, wherein the patient was previously administered imatinib.

18. The method of any one of claims 1-17, wherein the KIT driven melanoma has a baseline mutation selected from the group consisting of a KIT exon 9 mutation, a KIT exon 11 mutation, a KIT exon 13 mutation, a KIT exon 17 mutation, and a KIT exon 18 mutation.

19. The method of any one of claims 1-18, wherein the KIT driven melanoma is caused by overexpression of wild-type KIT.

20. The method of any one of claims 1-19, wherein, after at least one 28-day cycle, the patient has a progression-free survival as measured using RECIST 1.1.

21. A method of treating melanoma in a patient in need thereof, comprising orally administering to the patient 100 mg to 600 mg of ripretinib daily, and one or more additional therapeutic agents.

22. The method of claim 21, comprising orally administering to the patient 100 mg to 300 mg of ripretinib daily.

23. The method of claim 21 or 22, comprising orally administering to the patient 150 mg of ripretinib daily.

24. The method of any one of claims 21-23, wherein the melanoma is a KIT driven melanoma.

25. The method of any one of claims 21-24, wherein the KIT driven melanoma has a baseline mutation selected from the group consisting of a KIT exon 9 mutation, a KIT exon 11 mutation, a KIT exon 13 mutation, a KIT exon 17 mutation, and a KIT exon 18 mutation.

26. The method of claim 24 or 25, wherein the KIT driven melanoma is caused by overexpression of wild-type KIT.

27. The method of any one of claims 1-26, wherein the melanoma is selected from the group consisting of cutaneous melanoma and noncutaneous melanoma.

28. The method of claim 27, wherein the cutaneous melanoma is selected from the group consisting of superficial spreading melanoma, nodular melanoma, acral-lentiginous melanoma, amelanotic melanoma, and desmoplastic melanoma.

29. The method of claim 28, wherein the noncutaneous melanoma is selected from ocular melanoma and mucosal melanoma.
